(19) United States
(12) Patent Application Publication
(10) Pub. No.: US 2025/0388637 A1
(43) Pub. Date: Dec. 25, 2025

(54) PDGF-D PRODOMAIN AND ITS MUTANT AND FUSION

(71) Applicant: TIANJIN UNIVERSITY, Tianjin (CN)

(72) Inventors: Lizhi MI, Tianjin (CN); Xiaohong QIN, Tianjin (CN); Linli LI, Tianjin (CN); Dan WU, Tianjin (CN); Jianyu ZHOU, Tianjin (CN)

(73) Assignee: TIANJIN UNIVERSITY, Tianjin (CN)

(21) Appl. No.: 18/879,260

(22) PCT Filed: Jun. 29, 2023
(86) PCT No.: PCT/CN2023/104050

§ 371 Date: Dec. 27, 2024

(30) Foreign Application Priority Data
Jun. 29, 2022 (WO) PCT/CN2022/102564

(51) Int. Cl. C07K 14/49

(52) U.S. Cl. CPC C07K 14/49; C07K 2319/00

(57) ABSTRACT

The present application is directed to PDGF-D prodomain or its mutant or fusion. The present application is also directed to a process or use of the PDGF-D prodomain or its mutant or fusion for inhibiting PDGFR phosphorylation, for inhibiting cell proliferation stimulated by PDGF, or for preventing and/or treating diseases associated therewith.

### TECHNICAL FIELD

The present application is directed to PDGF-D prodomain or its mutant or fusion. The present application is also directed to a process or use of the PDGF-D prodomain or its mutant or fusion for inhibiting PDGFR phosphorylation, for inhibiting cell proliferation stimulated by PDGF, or for preventing and/or treating diseases associated therewith.

BACKGROUND

PDGF-D is a growth factor regulating blood vessel development, wound healing, and organogenesis [1-3]. It is highly expressed in heart, pancreas, and ovary [2]. Abnormal functions of PDGF-D are associated with progressive renal diseases, cancers, and fibrosis [4-7]. Therefore, better understanding the biology of PDGF-D signaling and developing therapeutics to intervene PDGF-D dysfunctions are of great significance.

PDGF-D belongs to PDGF/VEGF growth factor family [2, 3]. These growth factors are co-evolved from a common ancestor in invertebrates and become diverged into two subfamilies (PDGF and VEGF, respectively) in vertebrates [1, 8]. All family members share a structurally conserved cysteine-knot core, which constitute the mature, active form of the growth factors [9]. In addition, all of them are involved in the regulation of angiogenesis in development and blood vessel homeostasis in adult [1].

In PDGF subfamily, PDGF-A and PDGF-B are secreted in an active form, freely diffuse in extracellular space, or bind to extracellular matrix via their C-terminal retention signal sequences [1]. In contrast, PDGF-C and PDGF-D are synthesized and secreted in a latent, inactive complex referred to as pro-complex, in which the prodomain is covalently linked to the growth factor domain via a peptide-bond (FIG. S1). As such, both PDGF-C and PDGF-D pro-complexes need to be proteolytically activated by tissue plasminogen activator (tPA) and urokinase plasminogen activator (uPA), respectively, to release their growth factor domains [2, 3, 10-12]. In addition, both pro-complexes can also be cleaved and activated by matriptase [13, 14].

Besides the hinge domain identified in the prodomains of PDGF-A and PDGF-B, the prodomains of PDGF-C and PDGF-D contain an additional CUB domain, which confers the latency of these two growth factors [3, 12]. However, beyond the known mechanism for the proteolytic activation, the following questions remain largely unknown: how does the CUB domain interact with the growth factor in the pro-complexes of PDGF-C and PDGF-D? How do these interactions contribute to the latency and activation of these two growth factors? In line with these mysteries, it had been reported that PDGF-C and PDGF-D genes can be transcribed into different splicing isoforms, in which two PDGF-C isoforms and one PDGF-D isoforms encode only their prodomains [15, 16]. The biological functions of these splicing isoforms remain unclear as conflicting results had been reported [12, 16].

To exert their biological functions, PDGF-D and its siblings are assembled into homo-or hetero-dimers and are recognized by their cognate receptors, including PDGFR-β and PDGFR-α, two receptor tyrosine kinases [1]. Among PDGF growth factor dimers, both PDGF-D and PDGF-B homodimers are recognized by the PDGFR-β, and thus share redundant functions in vascular development [3, 17, 18].

Apart from PDGFR-β, non-canonical receptor and co-receptor have been identified for the recognition of PDGF-D [7, 19, 20]. NKp44 is a PDGF-D receptor expressed on the surface of natural killer (NK) cells, innate lymphoid cell-1 and innate lymphoid cell-3 cells [7]. The recognition of PDGF-D by NKp44 could provoke the innate immunity of NK cells in tumor microenvironment [19]. On the other hand, neuropilin-1 is a newly-identified co-receptor for PDGF-D [20]. It also recognizes heparan sulfate and VEGFs [21]. The binding of PDGF-D to neuropilin-1 is involved in the cellular interactions between endothelial cells and pericytes [20].

### SUMMARY OF THE PRESENT APPLICATION

### The First Aspect of the Present Application

In the first aspect, the present application provides PDGF-D prodomain or its mutant or fusion, wherein the PDGF-D prodomain has the protein sequence of

RRDETIQVKGNGYVQSPRFPNSYPRNLLLTWRLHSQENTRIQLVFDNQF
GLEEAENDICRYDFVEVEDISETSTIIRGRWCGHKEVPPRIKSRTNQIK
ITFKSDDYFVAKPGFKIYYSLLEDFQPAAASETNWESVTSSISGVSYNS
PSVTDPTLIADALDKKIAEFDTVEDLLKYFNPESWQEDLENMYLDTPRY.

In another embodiment of the first aspect, the PDGF-D prodomain or its mutant is in the monomer or oligomer form, preferably the oligomer form.

In another embodiment of the first aspect, the mutant comprises 1, 2, 3, 4, 5 or 6 substitutions, deletions or insertions of amino acid in the protein sequence of the PDGF-D prodomain. In another embodiment of the first aspect, the mutant comprises one or more of the mutations 161-DKK/AAA-163, E37R, E71R and R93E relative to the PDGF-D prodomain.

In another embodiment of the first aspect, the fusion comprises a multi-valent protein in addition to the PDGF-D prodomain. In another embodiment of the first aspect, the multi-valent protein is fused to the N-terminus of the PDGF-D prodomain. In another embodiment of the first aspect, the multi-valent protein is GCN4 protein.

### The Second Aspect of the Present Application

In the second aspect, the present application provides a process or use of the PDGF-D prodomain or its mutant or fusion as defined in the first aspect (including each of the embodiments of the first aspect) for inhibiting PDGFR phosphorylation.

In another embodiment of the second aspect, the use includes use of the PDGF-D prodomain or its mutant or fusion as defined in the first aspect (including each of the embodiments of the first aspect) in manufacture of a kit or medicament for inhibiting PDGFR phosphorylation.

In another embodiment of the second aspect, the PDGFR is PDGFR-β. In another embodiment of the second aspect, the phosphorylation is mediated by PDGF-B or PDGF-D. In another embodiment of the second aspect, the PDGF-D is in the activated form.

### The Third Aspect of the Present Application

In the third aspect, the present application provides a process or use of PDGF-D prodomain or its mutant or fusion as defined in the first aspect (including each of the embodiments of the first aspect) for inhibiting cell proliferation stimulated by PDGF.

In another embodiment of the third aspect, the use includes use of the PDGF-D prodomain or its mutant or fusion as defined in the first aspect (including each of the embodiments of the first aspect) in manufacture of a kit or medicament for inhibiting cell proliferation stimulated by PDGF.

In another embodiment of the third aspect, the cell proliferation is stimulated by PDGF-B or PDGF-D. In another embodiment of the second aspect, the PDGF-D is in the activated form.

In another embodiment of the third aspect, the cell is the one expressing PDGFR. In another embodiment of the third aspect, the cell is a renal cell (e.g., BHK-21) or a fibroblast cell (e.g., NIH 3T3). In another embodiment of the third aspect, the PDGFR is PDGFR-β.

In another embodiment of the third aspect, when the PDGFR is PDGFR-β, the cell proliferation is stimulated by PDGF-B and PDGF-D, and when the PDGFR is other than PDGFR-β, the cell proliferation is stimulated by PDGF-D.

### The Fourth Aspect of the Present Application

In the fourth aspect, the present application provides a process or use of PDGF-D prodomain or its mutant or fusion as defined in the first aspect (including each of the embodiments of the first aspect) for preventing and/or treating diseases associated with PDGFR phosphorylation or cell proliferation stimulated by PDGF.

In another embodiment of the fourth aspect, the use includes use of the PDGF-D prodomain or its mutant or fusion as defined in the first aspect (including each of the embodiments of the first aspect) in manufacture of a kit or medicament for preventing and/or treating diseases associated with PDGFR phosphorylation or cell proliferation stimulated by PDGF.

In another embodiment of the fourth aspect, the PDGFR phosphorylation is as defined in the second aspect (including each of the embodiments of the second aspect). In another embodiment of the fourth aspect, the cell proliferation is as defined in the third aspect (including each of the embodiments of the third aspect).

In another embodiment of the fourth aspect, the diseases associated with PDGFR phosphorylation or cell proliferation stimulated by PDGF include atherosclerosis, fibrosis and tumors (e.g., malignant tumors).

The present application may be embodied in any other forms without departing from the spirit or scope thereof. The present application encompasses any and all combinations of the above aspects and embodiments. It is to be understood that any embodiment may be combined with any other embodiment(s) to describe an additional embodiment. It is also to be understood that an individual element from any embodiment may be combined with any and all other elements from any other embodiment(s) to describe an additional embodiment.

The Fifth Aspect of the Present Application

In the fifth aspect, the present application provides a mutated PDGF-D, wherein the mutation(s) exists in the CUB domain of the WT PDGF-D, the uPA cleavage site of the WT PDGF-D or the both. In the fifth aspect, the present application also provides a process or use of the mutated PDGF-D for stimulating cell proliferation.

In another embodiment of the fifth aspect, the mutation(s) is selected from the group consisting of E87R, E121R and R134E mutations on the CUB domain of the WT PDGF-D. In another embodiment of the fifth aspect, the mutation(s) is the one in which the fragment “RGRS” in the WT PDGF-D is substituted with the fragment “GAGA”. In another embodiment of the fifth aspect, the cell proliferation is intended for cell culture. In another embodiment of the fifth aspect, the cell is a NK cell.

In another embodiment of the fifth aspect, the use includes use of the mutated PDGF-D in manufacture of a kit or medicament for stimulating cell proliferation.

### SUMMARY OF THE FIGURES

FIG. 1. PDGF-D prodomain inhibited the PDGFR-β phosphorylation stimulated with PDGF-B.

A) Diagram of the primary structures of PDGF-D prodomain (aa. 51-246), hinge (aa. 197-246), and PDGF-D^{DKK/AAA} prodomain (aa. 51-246, 211-DKK/AAA-213). An MBP tag followed by a Hisx6 tag, a Flag tag, and a 3C cleavage site was introduced at the N-termini of these proteins.

B) The purification of MBP-tagged PDGF-D Prodomain. Affinity-purified MBP-Prodomain was loaded onto a Hitrap Q column equilibrated with buffer A (20 mM Tris-HCl, pH 8.0). The bound proteins were fractionated with a linear gradient of NaCl from 0 M to 1M concentration. Two fractions containing MBP-Prodomain were collected separately and subsequently purified with a Superdex G200 column equilibrated with 20 mM Tris-HCl (pH 8.0) and 150 mM NaCl. The fractionated samples from the gel-filtration column were subjected to non-reducing SDS-PAGE electrophoresis and Coomassie-blue staining.

C) The purification of MBP-tagged PDGF-D hinge domain. Affinity-purified MBP-Hinge was sequentially purified with a Hitrap Q column and a superdex G200 column. The gel-filtration chromatography profile and the Coomassie-blue-stained SDS-PAGE of fractionated samples were shown.

D) Purified PDGF-D Prodomain inhibited PDGF-B and PDGF-D stimulated PDGFR-β phosphorylation. BHK-21/PDGFR-β cells were starved in DMEM media for 20 hours and then stimulated for 15 minutes with either 4 nM purified PDGF-B or conditioned media from BHK-21 cells transiently transfected with PDGF-D or PDGE-D^{RGRS/GAGA}. At the same time of stimulation, 64 nM purified PDGF-D MBP-Prodomain oligomers or monomers were added to inhibit the stimulated PDGFR-β phosphorylation. After treatment, the cells were collected, lysed, and analyzed by western blotting. The expression and phosphorylation of PDGFR-β were detected with anti-protein C, pY740, and 4G10 antibodies, respectively.

E) Comparison of the inhibitory activities of the oligomeric and monomeric PDGF-D MBP-Prodomain on PDGF-B stimulated PDGFR-β phosphorylation. BHK-21/PDGFR-β cells were starved in DMEM media for 20 hours and then stimulated for 15minutes with 4 nM purified PDGF-B in the presence and absence of 64 nM purified MBP-Prodomain oligomers or monomers. The expression and phosphorylation of PDGFR-β were detected by western blotting using anti-protein C and 4G10 antibodies, respectively.

F) PDGF-D MBP-Prodomain inhibited PDGF-B stimulated PDGFR-βphosphorylation in a dose-dependent manner. BHK-21/PDGFR-β cells were starved in DMEM media for 20 hours and subsequently stimulated with 4 nM purified PDGF-B for 15 minutes. At the same time, PDGF-D MBP-Prodomain oligomers were diluted in a 2-fold serial dilution and were added at indicated concentrations to inhibit PDGFR-βphosphorylation. The relative phosphorylation levels of PDGFR-β were quantified from Western blotting results and were shown in panel.

G and H) PDGF-D prodomain directly bound to the PDGF-D but not PDGFR-βin MBP pull-down assay. MBP-tagged PDGF-D prodomain or control protein was individually incubated with the lysate of BHK-21/PDGFR-β cells (left) or the conditioned media from BHK-21 cells transiently transfected with the full-length PDGF-D (right). Both the PDGFR-β and the full-length PDGF-D were labeled with a protein C tag. The mixtures were subsequently incubated with MBP resin followed by repeated washing to remove unbound proteins. Then, the input and bound proteins were analyzed by western blotting using anti-protein C and anti-MBP antibodies.

I) In a dose-dependent experiment, PDGF-D MBP-Hinge hardly inhibited PDGF-B stimulated PDGFR-β phosphorylation. The experiment was performed as described in panel G.

J) Purified PDGF-D^{DKK/AAA} MBP-Prodomain also inhibited PDGF-B and PDGF-D stimulated PDGFR-β phosphorylation. The experiment was performed as described in panel D.

K) Mutations on the binding loops of the PDGF-D CUB domain had little effect on the biosynthesis of WT PDGF-D or PDGF-D^{RGRS/GAGA} mutant. Equal amount of the plasmids encoding the WT PDGF-D or indicated mutants were individually transiently-transfected into BHK-21 cells. The expressed PDGF-D proteins in the conditioned media were detected by western blotting using anti-protein C antibody.

L) Mutations on the predicted growth factor binding loops of the CUB domain enhanced the activity of PDGF-D^{RGRS/GAGA} in stimulating PDGFR-β phosphorylation, whereas the same mutations had less effect on the activity of WT PDGF-D. BHK-21/PDGFR-β cells were stimulated with the conditioned media from the cells transfected with the WT and mutated PDGF-Ds. The expression and phosphorylation of PDGFR-β were detected by western blotting using anti-protein C, pY740, and 4G10 antibodies, respectively.

FIG. 2. The purification of PDGF-D MBP-Prodomain^{DKK/AAA}

Affinity-purified MBP-Prodomain^{DKK/AAA} was loaded onto a Hitrap Q column equilibrated with buffer A (20 mM Tris-HCl, pH 8.0). The bound proteins were fractionated with a linear gradient of NaCl from 0 M to 1M concentration. Two fractions containing MBP-Prodomain^{DKK/AAA} were collected separately and subsequently purified with a superdex G200 column equilibrated with 20 mM Tris-HCl (pH 8.0), 150 mM NaCl. The fractionated samples from the gel-filtration column were subjected to SDS-PAGE electrophoresis and Coomassie blue staining.

FIG. 3. PDGF-D prodomain fused with GCN4 inhibited PDGF-B mediated PDGFR-β phosphorylation.

A) Diagram of the primary structure of GCN4-PDGF-D prodomain chimera. By adding GCN4 at the N-terminus of PDGF-D MBP-Prodomain, the prodomains will be linked into a multi-valent protein via the tetramerization of GCN4.

B) The purification of PDGF-D MBP-GCN4-Prodomain chimera. Affinity-purified MBP-GCN4-Prodomain was sequentially purified with an anion-exchange column and a superose 6 gel-filtration column. The gel-filtration chromatography profile and the non-reducing SDS-PAGE results of fractionated samples were shown.

D) Purified MBP-GCN4-Prodomain inhibited PDGF-B stimulated PDGFR-βphosphorylation in a dose-dependent manner. BHK-21/PDGFR-β cells were starved in DMEM media for 20 hours and subsequently stimulated with 4 nM purified PDGF-B for 15 minutes. At the same time, MBP-GCN4-Prodomain were diluted in a 2-fold serial dilution and were added at indicated concentrations to inhibit PDGFR-βphosphorylation. The expression and phosphorylation of PDGFR-β were detected with protein C and 4G10 antibodies, respectively.

E) The relative phosphorylation levels of PDGFR-β were quantified from Western blotting results in panel.

FIG. 4. PDGF-D prodomain differentially inhibits PDGF-B and PDGF-D stimulated NIH 3T3 cell and BHK-21/PDGFR-β cell proliferation.

A and B) NIH 3T3 (A) and BHK-21/PDGFR-β (B) cells were seeded into 96-well plates at a density of 3×10⁴ cells/well. The seeded cells were starved in DMEM for 20hours. Then the cells were stimulated with 2.5 nM purified PDGF-B or conditioned media from PDGF-D or PDGF-D^{RGRS/GAGA} transfected BHK-21 cells. At the same time of stimulation, cells were treated with oligomeric or monomeric PDGF-D prodomain at a concentration of 64 nM. The cell density was determined using the cell proliferation assay described in the method. The bar graphs represent the mean ±S. D. from three biological repeats. The statistical significance was determined as described in the method.

C and D) The inhibitory activities of PDGF-D^{DKK/AAA} prodomain on PDGF-B and PDGF-D stimulated NIH 3T3 (C) or BHK-21/PDGFR-β (D) cell proliferation. The experiments were performed as described in panel A.

### EXAMPLES

### Materials

Recombinant human PDGF-B was purchased from Arco (China, cat. #DDB-H4112). The plasmid encoding phospho-tyrosine antibody 4G10 and Flag antibody were constructed according to the literature. These antibodies were affinity-purified from transformed BL21 (DE3) cells and transfected HEK293 cells, respectively. PDGFR-β pY740 phospho-tyrosine antibody was purchased from Abmart (China, cat. #T55673). Protein C antibody was from Genscript Biotech. (China, cat. A01774). The goat-anti-rabbit and goat-anti-mouse antibodies were from Proteintech (China, cat. #SA00001-2/SW067A00160). DMEM was from Biological Industries (cat. #zc013-06-1005-57-1ACS). DMSO and fetal bovine serum (FBS) was from Sangon Biotech (China, cat. #E600001). Puromycin and MTT (3-(4, 5)-dimethylthiahiazo (-z-yl)-3, 5-di-phenytetrazoliumromide) was from Solarbio (China, cat. #A8020). PVDF membrane was purchased from Thermo Fisher (cat. #88518).

### General Procedures

### Plasmid Construction, Protein Expression and Purification

The full-length PDGF-D with a protein C tag at the C-terminus was cloned into the pTT5 vector. Various PDGF-D mutants were generated from the recombinant plasmid using DpnI-mediated site-directed mutagenesis (New England Biolabs, cat. #R0176S). All constructs used in this study were confirmed by DNA sequencing.

The genes encoding PDGF-D prodomain (aa 51-246), hinge (aa 197-246), PDGF-D^{DKK/AAA} prodomain, and GCN4-prodomain were individually subcloned into the pET vector. In this vector, a sequence encoding an MBP tag followed by a His×6 tag, a Flag tag, and a 3C protease cleavage site was inserted at the 5′ end of the subcloned gene. These plasmids were transformed into Rosetta gami2 E. coli cells for expressing recombinant proteins.

The E. coli cells were cultured in LB media to log phase at OD₆₀₀ of 0.8. Then the cells were induced with 100 μM IPTG at 20-30° C. for 12 hours to express the recombinant proteins. Cultured cells were harvested by centrifugation, lysed by French Press in a buffer containing 20 mM Tris-HCl (pH 8.0), 5 mM imidazole (pH 8.0), 300 mM NaCl, and cleared by centrifugation. Cleared lysate was loaded on a 3 mL Ni2+-NTA column, which was subsequently washed with 20 column volume (CV) of washing buffer containing 20 mM Tris-HCl (pH 8.0), 40 mM imidazole (pH 8.0), 300 mM NaCl, and eluted with 20 mM Tris-HCl (pH 8.0), 300 mM imidazole (pH 8.0), 300 mM NaCl. The yield and purity of purified proteins were analyzed with SDS-PAGE electrophoresis and UV absorption.

After affinity purification, recombinant proteins were further purified by anion-exchange chromatography (Hitrap Q column) and gel-filtration chromatography. In anion-exchange chromatography, the column was equilibrated with buffer A (20 mM Tris-HCl, pH 8.0) and bound proteins were fractionated with a linear gradient of NaCl from 0 M to 1M concentration. In gel-filtration chromatography, either a superdex G200 or superose 6 column was used and equilibrated with a buffer containing 20 mM Tris-HCl (pH 8.0) and 150 mM NaCl.

The proteins involved in the present application, including PDGF-D, PDGF-D^{RGRS/GAGA} MBP-Prodomain, MBP-Prodomain^{DKK/AAA}, MBP-Hinge and MBP-GCN4-Prodomain, have the sequences as shown below:

PDGF-D:
MHRLIFVYTLICANFCSCRDTSATPQSASIKALRNANLRRDESNHLTDLYR
RDETIQVKGNGYVQSPRFPNSYPRNLLLTWRLHSQENTRIQLVFDNQFGLEEA
ENDICRYDFVEVEDISETSTIIRGRWCGHKEVPPRIKSRTNQIKITFKSDDYFVA
KPGFKIYYSLLEDFQPAAASETNWESVTSSISGVSYNSPSVTDPTLIADALDKKI
AEFDTVEDLLKYFNPESWQEDLENMYLDTPRYRGRSYHDRKSKVDLDRLND
DAKRYSCTPRNYSVNIREELKLANVVFFPRCLLVQRCGGNCGCGTVNWRSCT
CNSGKTVKKYHEVLQFEPGHIKRRGRAKTMALVDIQLDHHERCDCICSSRPPR
PDGF-D^{RGRS/GAGA}:
MHRLIFVYTLICANFCSCRDTSATPQSASIKALRNANLRRDESNHLTDLYR
RDETIQVKGNGYVQSPRFPNSYPRNLLLTWRLHSQENTRIQLVFDNQFGLEEA
ENDICRYDFVEVEDISETSTIIRGRWCGHKEVPPRIKSRTNQIKITFKSDDYFVA
KPGFKIYYSLLEDFQPAAASETNWESVTSSISGVSYNSPSVTDPTLIADALDKKI
AEFDTVEDLLKYFNPESWQEDLENMYLDTPRYGAGAYHDRKSKVDLDRLND
DAKRYSCTPRNYSVNIREELKLANVVFFPRCLLVQRCGGNCGCGTVNWRSCT
CNSGKTVKKYHEVLQFEPGHIKRRGRAKTMALVDIQLDHHERCDCICSSRPPR
MBP-Prodomain:
MKIEEGKLVIWINGDKGYNGLAEVGKKFEKDTGIKVTVEHPDKLEEKFP
QVAATGDGPDIIFWAHDRFGGYAQSGLLAEITPDKAFQDKLYPFTWDAVRYNG
KLIAYPIAVEALSLIYNKDLLPNPPKTWEEIPALDKELKAKGKSALMENLQEPY
FTWPLIAADGGYAFKYENGKYDIKDVGVDNAGAKAGLTFLVDLIKNKHMNA
DTDYSIAEAAFNKGETAMTINGPWAWSNIDTSKVNYGVTVLPTFKGQPSKPFV
GVLSAGINAASPNKELAKEFLENYLLTDEGLEAVNKDKPLGAVALKSYEEELA
KDPRIAATMENAQKGEIMPNIPQMSAFWYAVRTAVINAASGRQTVDEALKDA
QTNSHMHHHHHHDYKDHDGDYKDHDIDYKDDDDKLEVLFQGPGSRRDETI
QVKGNGYVQSPRFPNSYPRNLLLTWRLHSQENTRIQLVFDNQFGLEEAENDIC
RYDFVEVEDISETSTIIRGRWCGHKEVPPRIKSRTNQIKITFKSDDYFVAKPGFKI
YYSLLEDFQPAAASETNWESVTSSISGVSYNSPSVTDPTLIADALDKKIAEFDT
VEDLLKYFNPESWQEDLENMYLDTPRY
MBP-Prodomain^{DKK/AAA}:
MKIEEGKLVIWINGDKGYNGLAEVGKKFEKDTGIKVTVEHPDKLEEKFP
QVAATGDGPDIIFWAHDRFGGYAQSGLLAEITPDKAFQDKLYPFTWDAVRYNG
KLIAYPIAVEALSLIYNKDLLPNPPKTWEEIPALDKELKAKGKSALMFNLQEPY
FTWPLIAADGGYAFKYENGKYDIKDVGVDNAGAKAGLTFLVDLIKNKHMNA
DTDYSIAEAAFNKGETAMTINGPWAWSNIDTSKVNYGVTVLPTFKGQPSKPFV
GVLSAGINAASPNKELAKEFLENYLLTDEGLEAVNKDKPLGAVALKSYEEELA
KDPRIAATMENAQKGEIMPNIPQMSAFWYAVRTAVINAASGRQTVDEALKDA
QTNSHMHHHHHHDYKDHDGDYKDHDIDYKDDDDKLEVLFQGPGSRRDETI
QVKGNGYVQSPRFPNSYPRNLLLTWRLHSQENTRIQLVFDNQFGLEEAENDIC
RYDFVEVEDISETSTIIRGRWCGHKEVPPRIKSRTNQIKITFKSDDYFVAKPGFKI
YYSLLEDFQPAAASETNWESVTSSISGVSYNSPSVTDPTLIADALAAAIAEFDT
VEDLLKYFNPESWQEDLENMYLDTPRY
MBP-Hinge:
MKIEEGKLVIWINGDKGYNGLAEVGKKFEKDTGIKVTVEHPDKLEEKFP
QVAATGDGPDIIFWAHDRFGGYAQSGLLAEITPDKAFQDKLYPFTWDAVRYNG
KLIAYPIAVEALSLIYNKDLLPNPPKTWEEIPALDKELKAKGKSALMENLQEPY
FTWPLIAADGGYAFKYENGKYDIKDVGVDNAGAKAGLTFLVDLIKNKHMNA
DTDYSIAEAAFNKGETAMTINGPWAWSNIDTSKVNYGVTVLPTFKGQPSKPFV
GVLSAGINAASPNKELAKEFLENYLLTDEGLEAVNKDKPLGAVALKSYEEELA
KDPRIAATMENAQKGEIMPNIPQMSAFWYAVRTAVINAASGRQTVDEALKDA
QTNSHMHHHHHHDYKDHDGDYKDHDIDYKDDDDKLEVLFQGPGSSPSVTD
PTLIADALDKKIAEFDTVEDLLKYFNPESWQEDLENMYLDTPRY
MBP-GCN4-Prodomain:
MKIEEGKLVIWINGDKGYNGLAEVGKKFEKDTGIKVTVEHPDKLEEKFP
QVAATGDGPDIIFWAHDRFGGYAQSGLLAEITPDKAFQDKLYPFTWDAVRYNG
KLIAYPIAVEALSLIYNKDLLPNPPKTWEEIPALDKELKAKGKSALMENLQEPY
FTWPLIAADGGYAFKYENGKYDIKDVGVDNAGAKAGLTFLVDLIKNKHMNA
DTDYSIAEAAFNKGETAMTINGPWAWSNIDTSKVNYGVTVLPTFKGQPSKPFV
GVLSAGINAASPNKELAKEFLENYLLTDEGLEAVNKDKPLGAVALKSYEEELA
KDPRIAATMENAQKGEIMPNIPQMSAFWYAVRTAVINAASGRQTVDEALKDA
QTNSHMHHHHHHDYKDHDGDYKDHDIDYKDDDDKLEVLFQGPGSGRMKQ
LEDKVEELLSKNYHLENEVARLKKLCGERGRRDETIQVKGNGYVQSPRFPNS
YPRNLLLTWRLHSQENTRIQLVFDNQFGLEEAENDICRYDFVEVEDISETSTIIR
GRWCGHKEVPPRIKSRTNQIKITFKSDDYFVAKPGFKIYYSLLEDFQPAAASET
NWESVTSSISGVSYNSPSVTDPTLIADALDKKIAEFDTVEDLLKYFNPESWQE
DLENMYLDTPRY
Phosphorylation assay

The BHK-21/PDGFR-β cells, in which PDGFR-β was stably transfected, was established as described and maintained in DMEM supplemented with 10% FBS and 10 μg/mL puromycin [33]. Before experiment, these cells were seeded in 12-well plates at a density of 6×10⁵ cells/well and starved in DMEM for 24 hours in 5% CO₂ at 37° C. Then the cells were treated with PDGF-D prodomain, hinge, PDGF-D^{DKK/AAA} prodomain at indicated concentrations for 15 minutes. At the same time, the cells were stimulated with or without 4 nM PDGF-B reconstituted in 100 mM glacial acetic acid or conditioned media from BHK-21 cells transiently transfected with PDGF-D or PDGF-D^{RGRS/GAGA}. Afterwards, the cells were lysed with RIPA (0.1% SDS, 150 mM NaCl, 1% Triton X-100, 10 mM EDTA, 50 mM Tris-HCl, pH 7.5, 2 mM PMSF, 1 mM Na₃VO₄); and the lysate was subjected to SDS-PAGE electrophoresis. The expression and phosphorylation of PDGFR-β were detected by western blotting using protein C antibody, pY740 phospho-PDGFR-β antibody, and 4G10 antibody, respectively.

MBP pull-down assay

Recombinant MBP-Prodomain or MBP-Control protein was expressed in Rosetta gami2 E. coli cells. PDGFR-β or PDGF-D with a protein C tag was expressed in BHK-21 cells as described. 650 μg of MBP-Prodomain or MBP-Control protein were incubated with 8 mL lysate of BHK-21/PDGFR-β cells overnight at 4° C. in binding buffer containing 150 mM NaCl, 1% Triton X-100, 10 mM EDTA, 50 mM Tris-HCl, pH 7.5, 2 mM PMSF, and 1 mM Na₃VO₄. Then, the mixture was incubated with MBP beads (Smart-Lifesciences, SA026GC01) for 4 hours. After extensive washing, bound proteins were analyzed by SDS-PAGE and Western blotting using anti-protein C antibody and anti-MBP antibody. For analyzing the binding of the prodomain to PDGF-D growth factor, 25 ml conditioned media from BHK-21 cells transiently transfected with full-length PDGF-D was incubated with 650 μg of MBP-Prodomain or MBP-Control protein, the binding assay was performed similarly as PDGFR-β.

### Cell Proliferation Assay

NIH 3T3 cells or BHK-21/PDGFR-β cells were seeded in 96-well plates at a density of 3×10⁴ cells/well. The seeded cells were starved in DMEM for 20 hours and subsequently treated with or without purified PDGF-B at 2.5 nM concentration or conditioned media from BHK-21 cells transiently transfected with PDGF-D or PDGF-D^{RGRS/GAGA}. At the same time, these cells were treated with or without purified PDGF-D prodomain oligomers or monomers at 64 nM concentration. Treated cells were cultured for another 48 hours at 37° C. Then, 100 μL MTT at 0.5 mg/mL concentration was added into each well of the plate. The plates were incubated at RT for 4 hours before adding 100 μL DMSO into each well to dissolve formazan. The plates were shaken at RT for 10 minutes. The cell density in each well was determined by measuring the optical density at 570 nm using EnSpire Multilabel reader.

### Example 1. PDGF-D Prodomain and a Mutant Thereof Were Used for Inhibiting PDGF-B and PDGF-D Mediated PDGFR-β Phosphorylation

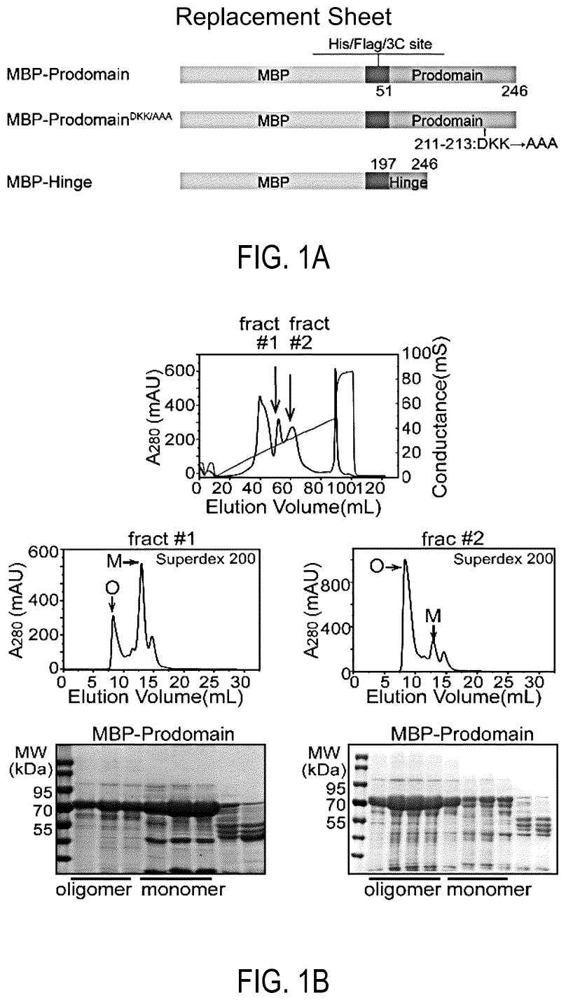

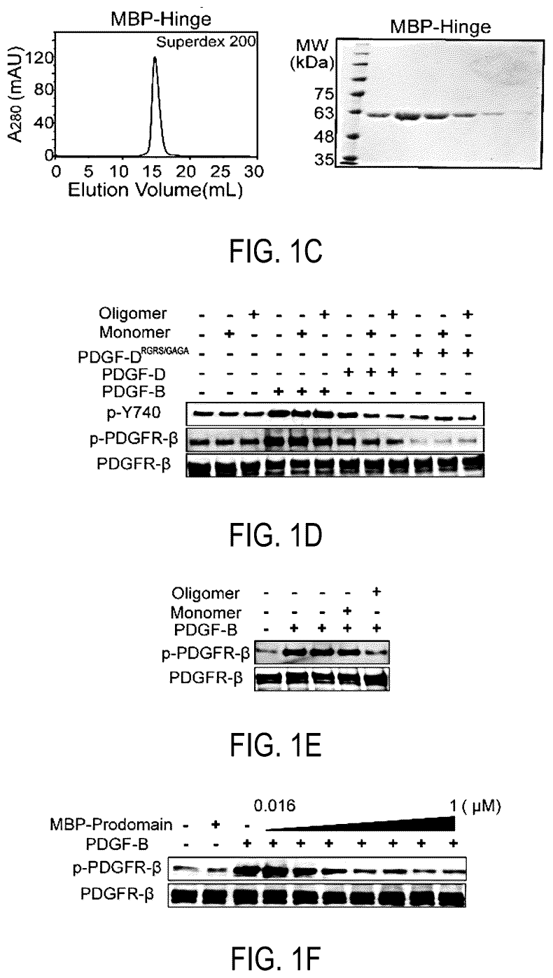

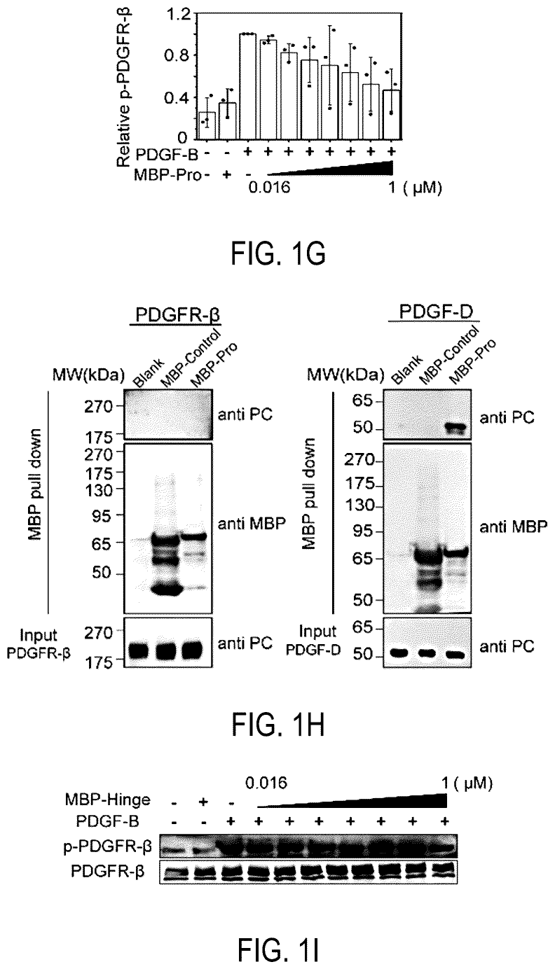

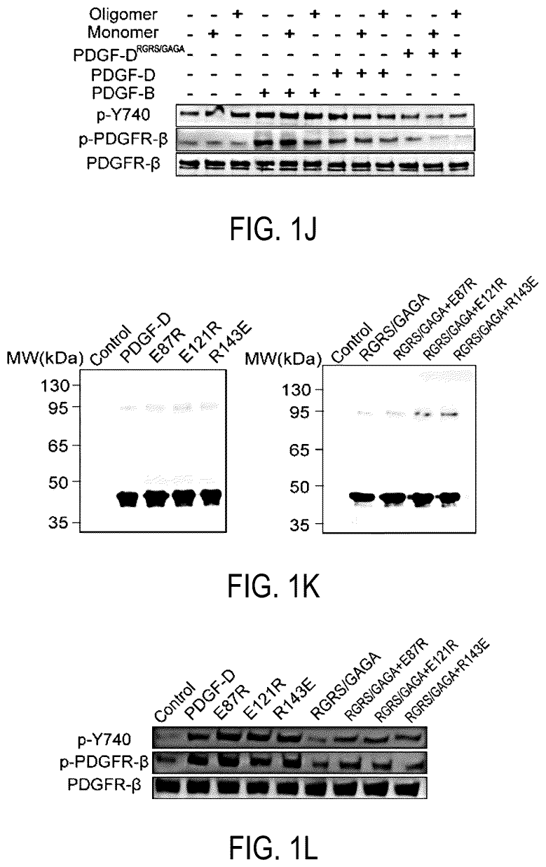

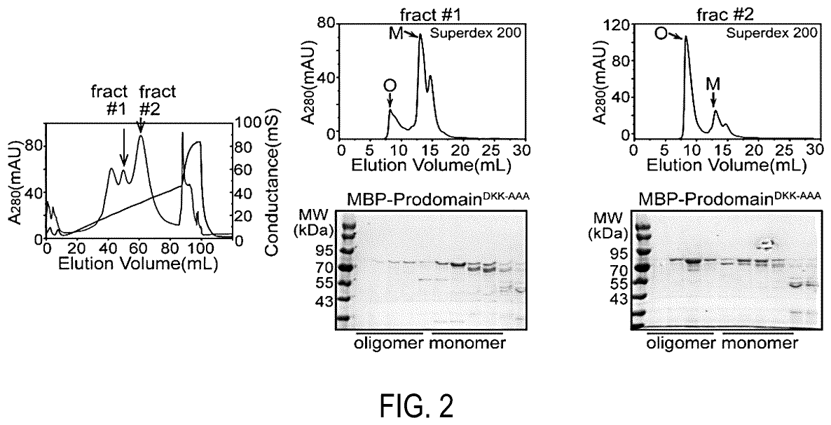

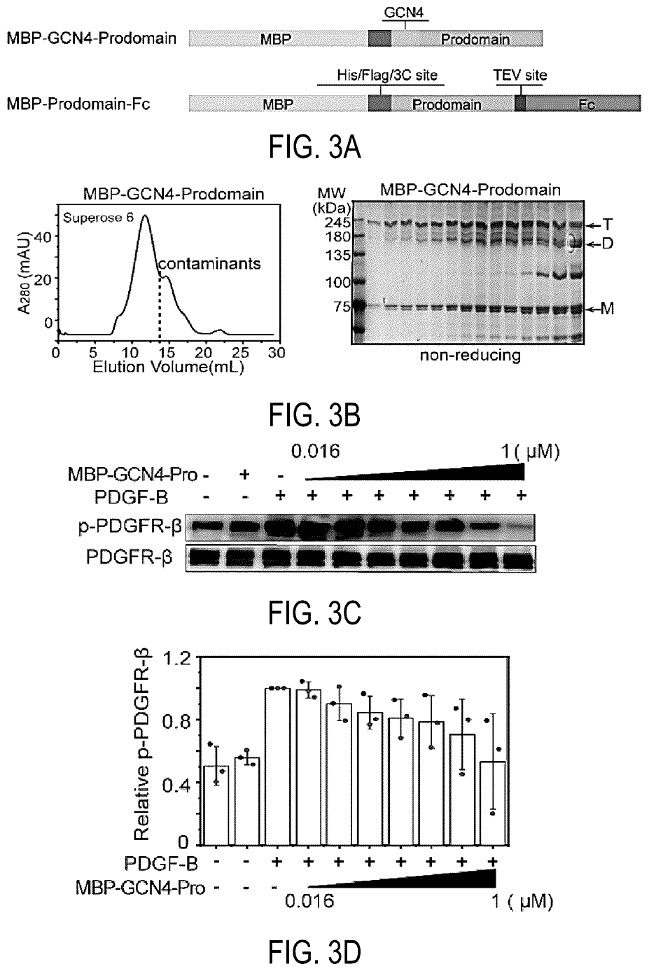

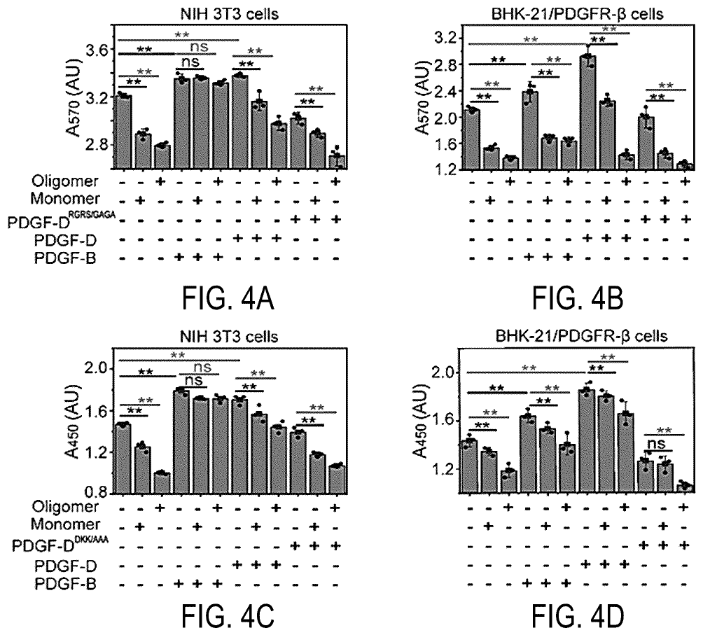

We expressed and purified MBP-tagged wild type (WT) PDGF-D prodomain (aa 51-246), the hinge (aa 197-246), and a prodomain mutant (PDGF-D^{DKK/AAA}) (FIG. 1A), and subsequently tested their inhibitory activities against PDGF-B or PDGF-D stimulated PDGFR-β phosphorylation.

The MBP-tagged WT PDGF-D prodomain (hereafter, it was referred to as MBP-Prodomain) was affinity-purified with a Ni²⁺-NTA column and subjected to reducing and non-reducing SDS-PAGE electrophoresis. As shown by the non-reducing SDS-PAGE results, MBP-Prodomain was detected as a 68.5 kDa protein without forming disulfide-linked dimer (FIG. 1B). The affinity-purified MBP-Prodomain was further purified with an anion-exchange chromatography (Hitrap Q) (FIG. 1B). Two different fractions containing MBP-Prodomain were eluted from the column at conductance of 27 mS and 34 mS, respectively. These fractions were independently analyzed with gel-filtration chromatography using a superdex G200 column. As shown in FIG. 1B, the first fraction eluted from Hitrap Q column contained a large fraction of monomeric MBP-Prodomain, whereas the second fraction from the Q column had more oligomeric MBP-Prodomain (FIG. 1B). Similar results were obtained when we purified the MBP-tagged PDGF-D^{DKK/AAA} prodomain mutant (FIG. 2). In addition, following the same protocol, we purified the MBP-tagged PDGF-D hinge domain. It was monodispersed in gel-filtration chromatography with an apparent molecular weight of 51.4 kDa (FIG. 1C).

We compared the inhibitory activities of these purified proteins. The MBP-Prodomain inhibited PDGF-B as well as PDGF-D stimulated PDGFR-βautophosphorylation in a dose-dependent manner. Especially, the oligomeric MBP-Prodomain was more potent than the monomeric MBP-Prodomain in the inhibition of PDGFR-β phosphorylation (FIGS. 1D, 1E). With increasing concentrations of the oligomeric MBP-Prodomain, the stimulated phosphorylation level of PDGFR-β under 64 nM PDGF-B was gradually decreased to 60% of non-prodomain treated sample (FIGS. 1F, 1G).

To understand the underlining mechanism for the inhibitory activity of PDGF-D prodomain, we did a pull-down analysis to study whether the prodomain could directly bind to the receptor PDGFR-β or the growth factor PDGF-D. Purified MBP-Prodomain or MBP-Control protein was separately incubated with the lysate of BHK-21/PDGFR-β cells or the conditioned media from BHK-21 cells transiently transfected with PDGF-D. After incubation with MBP resin and extensive washing, the bound proteins were eluted and detected by Western blotting. As shown in FIG. 1H, MBP-prodomain only specifically pulled down PDGF-D but not PDGFR-β. Combined with the inhibitory activities of the prodomain, our results indicated that the prodomain binds back to PDGF-D to prevent the growth factor from stimulating PDGFR-β phosphorylation.

Then, we studied how the hinge and CUB domains of PDGF-D contribute to the inhibitory activities of the prodomain. As it was shown in FIG. 1I, the hinge by itself had very low inhibitory activity toward PDGF-B stimulated PDGFR-β phosphorylation. The inhibitory effect only was detected at 1 μM concentration of the hinge. On the other hand, introducing mutation DKK/AAA at the hinge of the prodomain also impaired the MBP-Prodomain from inhibiting PDGF-B and PDGF-D stimulated PDGFR-βphosphorylation (FIG. 1J). These data collectively indicate that the hinge and CUB domain work together to coordinately inhibit the growth factor from recognition and stimulation of PDGFR-β.

We mutated three conserved residues, one from each loop of the three loops on the CUB domain which were presumed to be involved in the binding to the PDGF-D growth factor, for analyzing their impacts on the biosynthesis and activity of the growth factor. All of these mutations had little effect on the biosynthesis of WT PDGF-D or uncleavable, latent PDGF-D^{RGRS/GAGA} mutant (FIG. 1K). However, E87R, E121R and R134E mutations on the CUB domain enhanced the activity of PDGF-D^{RGRS/GAGA} in stimulating PDGFR-β phosphorylation, whereas the same mutations introduced on the WT PDGF-D had only mild effects on stimulating PDGFR-β phosphorylation (FIG. 1L). This result suggests that interfering the interactions between the CUB domain and the growth factor domain could partially relieve the inhibitory actions of the prodomain.

### Example 2. PDGF-D Prodomain Fused With GCN4 Were Used for Inhibiting PDGF-B Mediated PDGFR-β Phosphorylation

As the oligomeric PDGF-D prodomain was more potent than the monomeric prodomain in inhibiting the growth factor activity, we tested whether fusion of the prodomain into a multi-valent protein could enhance the inhibitory activity of the prodomain.

We fused the GCN4 protein to the N-terminus of the prodomain (FIG. 3A) and purified the chimeras subsequently with affinity and anion-exchange chromatography. Then, we analyzed the samples with gel-filtration chromatography using a superose 6 column (FIGS. 3B-E). As it was shown in FIGS. 3B-E, GCN4 fused PDGF-D prodomain was eluted from the column in a single but broad peak, indicating that these samples are conformationally heterogenous.

In PDGFR-β phosphorylation assay, we found N-terminally fused GCN4-prodomain inhibited PDGF-B stimulated PDGFR-β phosphorylation in a dose-dependent manner (FIGS. 3D, 3E, S2). However, the potency of the chimera was lower than that of the oligomeric MBP-Prodomain. At 64 nM concentration of the chimera, the phosphorylation level of PDGFR-β was only decreased by 16%, while, at the same concentration, the oligomeric MBP-Prodomain reduced the phosphorylation of PDGFR-β by 40% (FIGS. 1G, 3E).

### Example 3. PDGF-D Prodomain and a Mutant Thereof Were Used for Inhibiting PDGF-B and PDGF-D Stimulated Proliferation of NIH 3T3 and BHK-21/PDGFR-β Cells

To further investigate the functions of PDGF-D prodomain, we examined its inhibitory activities on the proliferation of fibroblast cells. To our surprise, we found PDGF-D prodomain exhibited differential inhibition on PDGF-B and PDGF-D mediated cell proliferation.

As it was shown in FIG. 4A, both purified PDGF-B and conditioned media from PDGF-D transfected cells stimulated the proliferation of NIH 3T3 cells. In contrast, the conditioned media from the cells transfected with uPA cleavage site mutant PDGF-D^{RGRS/GAGA} was not able to stimulate the proliferation of NIH 3T3 cells. Adding PDGF-D MBP-Prodomain, either in monomeric or oligomeric state, inhibited the proliferation of cells treated with none, PDGF-D, or PDGF-D^{RGRS/GAGA}, indicating PDGF-D prodomain could inhibit the endogenous as well as autocrine PDGF-D signals.

However, PDGF-D MBP-Prodomain failed to inhibit the proliferation of NIH 3T3 cells treated with PDGF-B (FIG. 4A). To study whether this difference was associated with PDGFR-β signaling, we stably transfected PDGFR-β into BHK-21 cells, in which the endogenous PDGFR-β expression is low. In PDGFR-β transfected BHK-21 cells, PDGF-D MBP-Prodomain inhibited PDGF-B mediated cell proliferation (FIG. 4B).

It was predicted that the DKK at the hinge of PDGF-D was a cleavage site for the protease PLpro (papain-like protease) [28]. Therefore, we studied whether sequential cleavage of the prodomain is critical for its inhibitory functions and whether mutation on this cleavage site can restore its inhibitory activity. As shown in FIG. 4C, PDGF-D^{DKK/AAA} prodomain was still not able to inhibit the proliferation of NIH 3T3 cells stimulated with PDGF-B, but it inhibited the proliferation of NIH 3T3 cells treated with PDGF-D. As the WT PDGF-D prodomain, PDGF-D^{DKK/AAA} prodomain inhibited the proliferation of BHK-21/PDGFR-β cells stimulated with PDGF-B and PDGF-D (FIG. 4D).

These results suggested other factors expressed by NIH 3T3 cells attributed to the desensitization of PDGF-D prodomain to PDGF-B stimulation.

To sum up, PDGD-D plays an important role in vascular development, cancer, and innate immunity [29]. In this study, we studied the inhibitory functions of PDGF-D MBP-Prodomain on PDGF-B and PDGF-D signaling.

Based on alphafold2 prediction and known biology [1-3, 22], we predicted that the CUB and hinge domains of PDGF-D bind at the opposite sides of the growth factor, and thus coordinately prevent the activation of the growth factor. We provided several PDGF-D MBP-Prodomain mutants and chimeras and compared their inhibitory activities on PDGF-B or PDGF-D stimulated PDGFR-β phosphorylation. Our biochemical data shown that the prodomain could bind back to the growth factor to prevent the stimulation of PDGFR-β. In addition, we found three residues at three different loops of CUB domain were involved in the inhibitory interactions with the growth factor. PDGF-D growth factor can be recognized by multiple receptors, including PDGFR-β, NKp44, and a co-receptor neuropilin-1 [7, 19, 20].

In our studies, we found purified PDGF-D MBP-Prodomain formed oligomers as well as monomers. The oligomeric MBP-Prodomain is more potent than the monomer in inhibiting PDGFR-β phosphorylation and fibroblast cell proliferation.

Both PDGF-B and PDGF-D are cognate ligands for PDGFR-β and share redundant functions in certain biological and pathological settings. In our studies, we found PDGF-D MBP-Prodomain cross-inhibited both PDGF-B and PDGF-D signaling in PDGFR-β phosphorylation as well as BHK-21 cell proliferation. However, differential inhibitory activities on PDGF-B and PDGF-D signaling were observed when we analyzed the inhibition of NIH 3T3 cell proliferation by PDGF-D MBP-Prodomain. PDGF-D MBP-Prodomain failed to inhibit the proliferation of NIH 3T3 cells stimulated with PDGF-B but not PDGF-D.

### REFERENCES

[1] Andrae J, Gallini R, Betsholtz C. Role of platelet-derived growth factors in physiology and medicine. GENES & DEVELOPMENT. 2008; 22:1276-312.
[2] LaRochelle W J, Jeffers M, McDonald W F, Chillakuru R A, Giese N A, Lokker N A, et al. PDGFD, a new protease-activated growth factor. NATURE CELL BIOLOGY. 2001; 3:517-21.
[3] Bergsten E, Uutela M, Li X R, Pietras K, Ostman A, Heldin C H, et al. PDGF-D is a specific, protease-activated ligand for the PDGF beta-receptor. NATURE CELL BIOLOGY. 2001; 3:512-6.
[4] Ostendorf T, van Roeyen C R, Peterson J D, Kunter U, Eitner F, Hamad A J, et al. A fully human monoclonal antibody (CR002) identifies PDGF-D as a novel mediator of mesangioproliferative glomerulonephritis. J Am Soc Nephrol. 2003; 14:2237-47.
[5] Hudkins K L, Gilbertson D G, Carling M, Taneda S, Hughes S D, Holdren M S, et al. Exogenous PDGF-D is a potent mesangial cell mitogen and causes a severe mesangial proliferative glomerulopathy. J Am Soc Nephrol. 2004; 15:286-98.
[6] Buhl E M, Djudjaj S, Babickova J, Klinkhammer B M, Folestad E, Borkham-Kamphorst E, et al. The role of PDGF-D in healthy and fibrotic kidneys. Kidney Int. 2016; 89:848-61.
[7] Barrow A D, Edeling M A, Trifonov V, Luo J Q, Goyal P, Bohl B, et al. Natural Killer Cells Control Tumor Growth by Sensing a Growth Factor. CELL. 2018; 172:534-+.
[8] Tarsitano M, De Falco S, Colonna V, McGhee J D, Persico M G. The C. elegans pvf-1 gene encodes a PDGF/VEGF-like factor able to bind mammalian VEGF receptors and to induce angiogenesis. FASEB J. 2006; 20:227-33.
[9] Sun P D, Davies D R. The cystine-knot growth-factor superfamily. Annu Rev Biophys Biomol Struct. 1995; 24:269-91.
[10] Ustach C V, Kim H R C. Platelet-derived growth factor D is activated by urokinase plasminogen activator in prostate carcinoma cells. MOLECULAR AND CELLULAR BIOLOGY. 2005; 25:6279-88.
[11] Fredriksson L, Li H, Fieber C, Li X, Eriksson U. Tissue plasminogen activator is a potent activator of PDGF-CC. EMBO JOURNAL. 2004; 23:3793-802.
[12] Li X R, Ponten A, Aase K, Karlsson L, Abramsson A, Uutela M, et al. PDGF-C is a new protease-activated ligand for the PDGF alpha-receptor. NATURE CELL BIOLOGY. 2000; 2:302-9.
[13] Najy A J, Won J J, Movilla L S, Kim H R. Differential tumorigenic potential and matriptase activation between PDGF B versus PDGF D in prostate cancer. Mol Cancer Res. 2012; 10:1087-97.
[14] Hurst N J, Jr., Najy A J, Ustach C V, Movilla L, Kim H R. Platelet-derived growth factor-C (PDGF-C) activation by serine proteases: implications for breast cancer progression. Biochem J. 2012; 441:909-18.
[15] Zhuo Y, Hoyle G W, Zhang J, Morris G, Lasky J A. A novel murine PDGF-D splicing variant results in significant differences in peptide expression and function. Biochem Biophys Res Commun. 2003; 308:126-32.
[16] Dijkmans J, Xu J, Masure S, Dhanaraj S, Gosiewska A, Geesin J, et al. Characterization of platelet-derived growth factor-C (PDGF-C): expression in normal and tumor cells, biological activity and chromosomal localization. Int J Biochem Cell Biol. 2002; 34:414-26.
[17] Hellstrom M, Kalen M, Lindahl P, Abramsson A, Betsholtz C. Role of PDGF-B and PDGFR-beta in recruitment of vascular smooth muscle cells and pericytes during embryonic blood vessel formation in the mouse. Development. 1999; 126:3047-55.
[18] Uutela M, Wirzenius M, Paavonen K, Rajantie I, He Y, Karpanen T, et al. PDGF-D induces macrophage recruitment, increased interstitial pressure, and blood vessel maturation during angiogenesis. Blood. 2004; 104:3198-204.
[19] Ma S B, Tang T T, Wu X J, Mansour A G, Lu T, Zhang J Y, et al. PDGF-D-PDGFR beta signaling enhances IL-15-mediated human natural killer cell survival. PROCEEDINGS OF THE NATIONAL ACADEMY OF SCIENCES OF THE UNITED STATES OF AMERICA. 2022; 119.
[20] Muhl L, Folestad E B, Gladh H, Wang Y X, Moessinger C, Jakobsson L, et al. Neuropilin 1 binds PDGF-D and is a co-receptor in PDGF-D-PDGFR beta signaling. JOURNAL OF CELL SCIENCE. 2017; 130:1365-78.
[21] Soker S, Takashima S, Miao H Q, Neufeld G, Klagsbrun M. Neuropilin-1 is expressed by endothelial and tumor cells as an isoform-specific receptor for vascular endothelial growth factor. Cell. 1998; 92:735-45.
[22] Jumper J, Evans R, Pritzel A, Green T, Figurnov M, Ronneberger O, et al. Highly accurate protein structure prediction with AlphaFold. Nature. 2021; 596:583-9.
[23] Altschul S F, Gish W, Miller W, Myers E W, Lipman D J. Basic local alignment search tool. J Mol Biol. 1990; 215:403-10.
[24] Leppänen V-M, Tvorogov D, Kisko K, Prota Andrea E, Jeltsch M, Anisimov A, et al. Structural and mechanistic insights into VEGF receptor 3 ligand binding and activation. Proceedings of the National Academy of Sciences. 2013; 110:12960-5.
[25] Hye-Ryong Shim A, Liu H, Focia Pamela J, Chen X, Lin P C, He X. Structures of a platelet-derived growth factor/propeptide complex and a platelet-derived growth factor/receptor complex. Proceedings of the National Academy of Sciences. 2010; 107: 11307-12.
[26] Cantoni C, Ponassi M, Biassoni R, Conte R, Spallarossa A, Moretta A, et al. The Three-Dimensional Structure of the Human NK Cell Receptor NKp44, a Triggering Partner in Natural Cytotoxicity. Structure. 2003; 11:725-34.
[27] Conway P, Tyka M D, DiMaio F, Konerding D E, Baker D. Relaxation of backbone bond geometry improves protein energy landscape modeling. Protein Sci. 2014; 23:47-55.
[28] Harrigan J A, Jacq X, Martin N M, Jackson S P. Deubiquitylating enzymes and drug discovery: emerging opportunities. NATURE REVIEWS DRUG DISCOVERY. 2018; 17:57-77.
[29] Ponten A, Folestad E B, Pietras K, Eriksson U. Platelet-derived growth factor D induces cardiac fibrosis and proliferation of vascular smooth muscle cells in heart-specific transgenic mice. CIRCULATION RESEARCH. 2005; 97:1036-45.
[30] Andersson M, Ostman A, Westermark B, Heldin C H. Characterization of the retention motif in the C-terminal part of the long splice form of platelet-derived growth factor A-chain. J Biol Chem. 1994; 269:926-30.
[31] Layton T B, Williams L, Yang N, Zhang M, Lee C, Feldmann M, et al. A vasculature niche orchestrates stromal cell phenotype through PDGF signaling: Importance in human fibrotic disease. Proc Natl Acad Sci U S A. 2022; 119: e2120336119.
[32] Kisseleva T, Brenner D. Molecular and cellular mechanisms of liver fibrosis and its regression. NATURE REVIEWS GASTROENTEROLOGY &HEPATOLOGY. 2021; 18:151-66.
[33] Shang Q, Zhao L, Wang X, Wang M, Sui S F, Mi L Z. Expression and purification of functional PDGF receptor beta. Biochem Biophys Res Commun. 2017; 489:353-9.

1. A PDGF-D prodomain or its mutant or fusion, wherein the PDGF-D prodomain has the protein sequence (SEQ. ID NO: 1) of
RRDETIQVKGNGYVQSPRFPNSYPRNLLLTWRLHSQENTRIQLVFDNQF
GLEEAENDICRYDFVEVEDISETSTIIRGRWCGHKEVPPRIKSRTNQIK
ITFKSDDYFVAKPGFKIYYSLLEDFQPAAASETNWESVTSSISGVSYNS
PSVTDPTLIADALDKKIAEFDTVEDLLKYFNPESWQEDLENMYLDTPRY.

2. A process or use of the PDGF-D prodomain or its mutant or fusion according to claim 1 for inhibiting PDGFR phosphorylation.

3. A process or use of PDGF-D prodomain or its mutant or fusion according to claim 1 for inhibiting cell proliferation stimulated by PDGF.

4. A process or use of PDGF-D prodomain or its mutant or fusion according to claim 1 for preventing and/or treating diseases associated with PDGFR phosphorylation or cell proliferation stimulated by PDGF.

5. A mutated PDGF-D, wherein the mutation(s) exists in the CUB domain of the WT PDGF-D, the uPA cleavage site of the WT PDGF-D or the both.

6. A process or use of the mutated PDGF-D for stimulating cell proliferation.


SEQUENCE LISTING (7 sequences)

SEQ ID NO: 1 - protein, 196 aa, synthetic construct
RRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRY

SEQ ID NO: 2 - protein, 371 aa, synthetic construct
MHRLIFVYTLICANFCSCRDTSATPQSASIKALRNANLRRDESNHLTDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRYRGRSYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPRC

SEQ ID NO: 3 - protein, 370 aa, synthetic construct
MHRLIFVYTLICANFCSCRDTSATPQSASIKALRNANLRRDESNHLTDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRYGAGAYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPR

SEQ ID NO: 4 - protein, 605 aa, synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSHMHHHHHHDYKDHDGDYKDHDIDYKDDDDKLEVLFQGPGSRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRY

SEQ ID NO: 5 - protein, 605 aa, synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSHMHHHHHHDYKDHDGDYKDHDIDYKDDDDKLEVLFQGPGSRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALAAAIAEFDTVEDLLKYFNPESWQEDLENMYLDTPRY

SEQ ID NO: 6 - protein, 459 aa, synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSHMHHHHHHDYKDHDGDYKDHDIDYKDDDDKLEVLFQGPGSSPSVTDPTLIADALDKKIAEFDTVEDLLKYFNPESWQEDLENMYLDTPRY

SEQ ID NO: 7 - protein, 640 aa, synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSHMHHHHHHDYKDHDGDYKDHDIDYKDDDDKLEVLFQGPGSGRMKQLEDKVEELLSKNYHLENEVARLKKLCGERGRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRY